(19) United States
(12) Patent Application Publication
(10) Pub. No.: US 2015/0120206 A1
(43) Pub. Date: Apr. 30, 2015

(54) CONSTITUTIONAL DISEASE PATTERN DETERMINATION METHOD AND APPARATUS

(71) Applicant: KOREA INSTITUTE OF ORIENTAL MEDICINE, Daejeon (KR)

(72) Inventors: Eun Su JANG, Daejeon (KR); Si Woo LEE, Jeonju-si (KR); Hee Jeong JIN, Daejeon (KR); Jong Hyang YOO, Daejeon (KR); Sang Oak DONG, Busan (KR)

(21) Appl. No.: 14/525,148

(22) Filed: Oct. 27, 2014

(30) Foreign Application Priority Data
Oct. 28, 2013 (KR) 10-2013-0128351

(51) Int. Cl. G06F 19/00; A61B 5/01; A61B 5/00; A61B 5/024; A61B 5/20

(52) U.S. Cl. CPC G06F 19/3431; A61B 5/024; A61B 5/20; A61B 5/4266; A61B 5/01; A61B 5/4255; A61B 5/4854
USPC 702/19

(57) ABSTRACT

Provided is a constitutional disease pattern determination apparatus including a calculator to calculate a first deficiency and excess index and a first cold and heat condition index of a user based on clinical information on the user, and a determiner to determine, based on a first reference table corresponding to a first constitution, a first constitutional disease pattern corresponding to the first deficiency and excess index and the first cold and heat condition index among a plurality of constitutional disease patterns included in the first reference table, wherein the first constitution is a constitution classified to correspond to the user based on a Sasang constitution.

### CROSS-REFERENCE TO RELATED APPLICATION

This application claims the priority benefit of Korean Patent Application No. 10-2013-0128351, filed on Oct. 28, 2013, in the Korean Intellectual Property Office, the disclosure of which is incorporated herein by reference.

### BACKGROUND

1. Field of the Invention

Embodiments of the present invention relate to a constitutional disease pattern determination method and apparatus, and more particularly, to a method and apparatus for determining a constitutional disease pattern based on a reference table corresponding to a constitution of a user by computing a deficiency and excess index and a cold and heat condition index of the user.

2. Description of the Related Art

Sasang constitutional medicine may classify constitutions into four types, a Taeyang type, a Taeeum type, a Soyang type, and a Soeum type. In the Sasang constitutional medicine, characteristics for each patient may be accepted and thus, an identical disease may be differently treated based on a constitution of a patient.

With respect to an identical disease, patterns may differ for each constitution. Also, an identical pattern may be a precursor of a disease or an indication of a healthy state based on a constitution. A constitutional disease pattern may refer to a discomfort that is differently analyzed for each constitution.

Although numerous medical specialists desire to apply the Sasang constitution to treatments for patients, in general, oriental medical doctors other than Sasang constitution specialists may have a difficulty in providing a treatment based on the Sasang constitution.

Accordingly, there is a desire for a system of determining a constitutional pattern disease to develop a treatment system based on the Sasang constitution.

### SUMMARY

According to an aspect of the present invention, there is provided a constitutional disease pattern determination apparatus including a calculator to calculate a first deficiency and excess index and a first cold and heat condition index of a user based on clinical information on the user, and a determiner to determine, based on a first reference table corresponding to a first constitution, a first constitutional disease pattern corresponding to the first deficiency and excess index and the first cold and heat condition index among a plurality of constitutional disease patterns included in the first reference table, wherein the first constitution is a constitution classified to correspond to the user based on a Sasang constitution.

The first reference table may correspond to user demographic information, and the user demographic information may include at least one of a gender and an age of the user.

The constitutional disease pattern determination apparatus may further include a selector to select the first reference table corresponding to the first constitution from among a plurality of reference tables corresponding to a plurality of constitutions classified based on the Sasang constitution.

The determiner may determine a constitutional disease pattern having a minimum geometric distance from a first point corresponding to the first deficiency and excess index and the first cold and heat condition index among the plurality of constitutional disease patterns included in the first reference table, as the first constitutional disease pattern.

The determiner may determine at least one second constitutional disease pattern having a greater geometric distance when compared to the first constitutional disease pattern among the plurality of constitutional disease patterns included in the first reference table, and provide the at least one second constitutional disease pattern.

The determiner may provide the first constitutional disease pattern and the at least one second constitutional disease pattern through an assignment of a weight, and the weight may be inversely proportional to the geometric distance.

The constitutional disease pattern determination apparatus may further include a verifier to verify the first constitution based on at least one of the clinical information and the demographic information on the user when identification information on the first constitution is absent.

The clinical information may include first data indicating a response of the user to each of a plurality of first medical examination items for calculating the first deficiency and excess index, and second data indicating a response of the user to each of a plurality of second medical examination items for calculating the first cold and heat condition index.

The first medical examination items may include at least one of an appetite state, a digestion state, cold sweats, bowel movement regularity, a number of nocturias, a sleep quality, a dry mouth, information on a pulse diagnosis, an asthenia examined from an abdominal diagnosis, and a subumbilical asthenia.

Based on the first data classified into a deficiency condition, an intermediate condition, and a excess condition, the calculator may calculate a deficiency condition score corresponding to a ratio of a difference between a number of excess conditions and a number of deficiency conditions, to a number of first medical examination items when the number of deficiency conditions is greater than the number of excess conditions, may calculate a excess condition score corresponding to a ratio of a difference between the number of deficiency conditions and the number of excess conditions, to the number of first medical examination items when the number of excess conditions is greater than the number of deficiency conditions, and may calculate an intermediate condition score when the number of deficiency conditions is equal to the number of excess conditions.

The second medical examination items may include at least one of an amount of sweat, a number of bowel movements, a feces shape, a number of urinations, a typical body temperature, a degree of cold sensitivity, a degree of heat sensitivity, an amount of drinking water, a drinking water temperature, a degree of thirst, information on a pulse diagnosis, and information on a tongue coating.

Based on the second data classified into a cold condition, an intermediate condition, and a heat condition, the calculator may calculate a cold condition score corresponding to a ratio of a difference between a number of heat conditions and a number of cold conditions, to a number of second medical examination items when the number of cold conditions is greater than the number of heat conditions, may calculate a heat condition score corresponding to a ratio of a difference between the number of cold conditions and the number of heat conditions, to the number of second medical examination items when the number of heat conditions is greater than the number of cold conditions, and may calculate an intermediate condition score when the number of cold conditions is equal to the number of heat conditions.

When the first constitution is classified as a Taeeum type based on the Sasang constitution, the plurality of constitutional disease patterns included in the first reference table may include at least one of an Supraspinal Exterior pattern, Esophagus Cold pattern, a Dryness Heat pattern, Dryness Heat pattern with Yin Blood Consumption symptoms, and an Exterior Interior combined pattern.

When the first constitution is classified as a Soeum type based on the Sasang constitution, the plurality of constitutional disease patterns included in the first reference table may include at least one of a depression-manic, a Yang collapse, a greater Yin pattern, a lesser Yin pattern, and an Exterior Interior combined pattern.

When the first constitution is classified as a Soyang type based on the Sasang constitution, the plurality of constitutional disease patterns included in the first reference table may include at least one of a Lesser Yang Wind Damage pattern, a Yin Depletion pattern, a Chest Heat congested pattern, a Yin Deficit Diurnal Heat pattern, and an Exterior Interior combined pattern.

When the first constitution is classified as a Taeyang type based on the Sasang constitution, the plurality of constitutional disease patterns included in the first reference table may include at least one of a Interior origin Small Intestine pattern accompanying Regurgitation, Interior origin Small Intestine pattern, and an Exterior Interior combined pattern.

According to another aspect of the present invention, there is also provided a method of determining a constitutional disease pattern of a user in a constitutional disease pattern determination apparatus, the method including calculating, by a calculator of the constitutional disease pattern determination apparatus, a first deficiency and excess index and a first cold and heat condition index of a user based on clinical information on the user, and determining, by a determiner of the constitutional disease pattern determination apparatus, a first constitutional disease pattern corresponding to the first deficiency and excess index and the first cold and heat condition index among a plurality of constitutional disease patterns included in a first reference table corresponding to a first constitution, based on the first reference table, wherein the first constitution is a constitution classified to correspond to the user based on a Sasang constitution.

The first reference table may correspond to user demographic information, and the user demographic information may include at least one of a gender and an age of the user.

The determining may include determining a constitutional disease pattern having a minimum geometric distance from a first point corresponding to the first deficiency and excess index and the first cold and heat condition index among the plurality of constitutional disease patterns included in the first reference table, as the first constitutional disease pattern.

The clinical information may include first data indicating a response of the user to each of a plurality of first medical examination items for calculating the first deficiency and excess index, and second data indicating a response of the user to each of a plurality of second medical examination items for calculating the first cold and heat condition index.

According to another aspect of the present invention, there is also provided a non-transitory computer-readable storage medium including a program including instructions to cause a computer to perform the method.

### BRIEF DESCRIPTION OF THE DRAWINGS

These and/or other aspects, features, and advantages of the invention will become apparent and more readily appreciated from the following description of exemplary embodiments, taken in conjunction with the accompanying drawings of which:

FIG. 1 is a block diagram illustrating a configuration of a constitutional disease pattern determination apparatus according to an example embodiment;

FIG. 2 is a flowchart illustrating a constitutional disease pattern determination method according to an example embodiment;

FIG. 3 is a diagram illustrating an operation of receiving clinical information of FIG. 2;

FIG. 4 is a diagram illustrating an example of medical examination information according to an example embodiment;

FIG. 5 is a diagram illustrating an operation of calculating a deficiency and excess index of FIG. 2;

FIG. 6 is a diagram illustrating an operation of calculating a cold and heat condition index of FIG. 2;

FIG. 7 is a diagram illustrating an operation of selecting a reference table corresponding to a user according to an example embodiment;

FIG. 8 is a diagram illustrating an example of selecting a reference table to correspond to user demographic information according to an example embodiment;

FIG. 9 is a diagram illustrating an operation of verifying a constitutional disease pattern of FIG. 2; and

FIG. 10 is a diagram illustrating an example of determining a constitutional disease pattern according to an example embodiment.

### DETAILED DESCRIPTION

Hereinafter, exemplary embodiments will be described in detail with reference to the accompanying drawings. However, it should be understood that these embodiments are not construed as limited thereto. Like reference numerals in the drawings denote like elements.

The terms used in this specification were selected to include current, widely-used, general terms, in consideration of the functions of the present invention. However, the terms may represent different meanings according to the intentions of the skilled person in the art or according to customary usage, the appearance of new technology, etc.

In certain cases, a term may be one that was arbitrarily established by the applicant. In such cases, the meaning of the term will be defined in the relevant portion of the detailed description. As such, the terms used in the specification are not to be defined simply by the name of the terms but are to be defined based on the meanings of the terms as well as the overall description of the present invention.

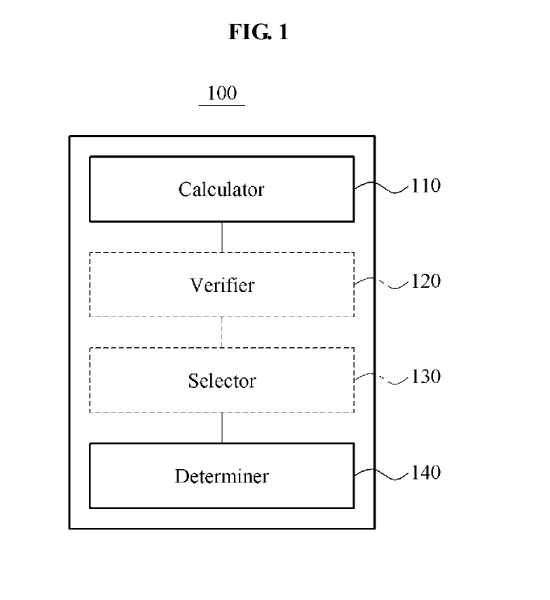

FIG. 1 is a block diagram illustrating a configuration of a constitutional disease pattern determination apparatus 100 according to an example embodiment.

Referring to FIG. 1, the constitutional disease pattern determination apparatus 100 may include a calculator 110, a verifier 120, a selector 130, and a determiner 140.

In an example embodiment, the constitutional disease pattern determination apparatus 100 may calculate a first deficiency and excess index and a first cold and heat condition index of a user based on user clinical information. The first deficiency and excess index and the first cold and heat condition index may be mapped to a first reference table. A result of the mapping may be used to determine a constitutional disease pattern. The first reference table may be may be obtained through a classification based on a Sasang constitution.

The calculator 110 may calculate the first deficiency and excess index and the first cold and heat condition index of the user based on the user clinical information.

In an example embodiment, the clinical information may include first data for calculating the first deficiency and excess index. The first data may include responses of the user to a plurality of first medical examination items.

For example, the first medical examination items may include at least one of an appetite state, a digestion state, cold sweats, bowel movement regularity, a nocturia frequency, a sleep quality, dry mouth, information on a pulse diagnosis, an asthenia examined from an abdominal diagnosis, and a subumbilical asthenia.

The first data may be recorded based on a predetermined numerical value and a predetermined term. As an example, clinical information on the cold sweats may be used to calculate the first cold and heat condition index. Based on a frequency of the cold sweats as a reference, a numerical value may be determined in advance. For example, “none” may be determined as “1”, “rarely” may be determined as “2”, “occasionally” may be determined as “3”, “often” may be determined as “4”, and “very often” may be determined as “5”. The numerical value may be determined in consideration of a state corresponding to the user.

The calculator 110 may classify the first data into a deficiency condition, an intermediate condition, and a excess condition. The calculator 110 may calculate the first deficiency and excess index based on a result of the classifying. The calculator 110 may count a number of deficiency conditions, a number of intermediate conditions, and a number of excess conditions.

When the number of deficiency conditions is greater than the number of excess conditions, the calculator 110 may calculate a deficiency condition score. The deficiency condition score may be calculated as a ratio of a difference between the number of deficiency conditions and the number of excess conditions to a number of first medical examination items.

When the number of excess conditions is greater than the number of deficiency conditions, the calculator 110 may calculate a excess condition score. The excess condition score may be calculated as a ratio of a difference between the number of excess conditions and the number of deficiency conditions to the number of first medical examination items.

When the number of deficiency conditions is equal to the number of excess conditions, the deficiency condition score and the excess condition score may be calculated as an intermediate condition score.

In another example embodiment, the clinical information may include second data for calculating the first cold and heat condition index. The second data may be responses of the user to a plurality of second medical examination items.

For example, the second medical examination items may include at least one of an amount of sweat, a number of bowel movements, a feces shape, a number of urinations, a typical body temperature, a degree of cold sensitivity, a degree of heat sensitivity, an amount of drinking water, a drinking water temperature, a degree of thirst, information on a pulse diagnosis, and information on a tongue coating.

The second data may be recorded based on a predetermined numerical value and a predetermined term. As an example, clinical information on the degree of cold sensitivity may be used to calculate the first cold and heat condition index. Based on the degree of cold sensitivity as a reference, the numerical value may be determined in advance. For example, “very low” may be determined to score one point, “low” may be determined to score two points, “intermediate” may be determined to score three points, “high” may be determined to score four points, and “very high” may be determined to score five points. The numerical value may be determined based on a state corresponding to the user.

The calculator 110 may classify the second data into a cold condition, an intermediate condition, and a heat condition. The calculator 110 may calculate the first cold and heat condition index based on a result of the classifying. The calculator 110 may count a number of cold conditions, a number of intermediate conditions, and a number of heat conditions.

When the number of cold conditions is greater than the number of heat conditions, the calculator 110 may calculate a cold condition score. The cold condition score may be calculated as a ratio of a difference between the number of cold conditions and the number of heat conditions to a number of the second medical examination items.

When the number of heat conditions is greater than the number of cold conditions, the calculator 110 may calculate a heat condition score. The heat condition score may be calculated as a ratio of a difference between the number of heat conditions and the number of cold conditions to the number of second medical examination items.

When the number of cold conditions is equal to the number of heat conditions, the cold condition score and the heat condition score may be calculated as an intermediate condition score.

When user constitutional information is absent, the verifier 120 may verify a constitution of the user based on at least one of the user clinical information and the user demographic information.

The Sasang constitution may classify constitutions into four types, a Taeyang type, a Taeeum type, a Soyang type, and a Soeum type. The constitution of the user may correspond to at least one of the Taeyang type, the Taeeum type, the Soyang type, and the Soeum type.

The selector 130 may select the first reference table corresponding to the constitution of the user from reference tables. A plurality of reference tables may be provided to correspond to a plurality of constitutions classified based on the Sasang constitution. For example, the reference tables may include a reference table for the Taeyang type, a reference table for the Taeeum type, a reference table for the Soyang type, and a reference table for the Soeum type based on the Sasang constitution. When the user corresponds to the Taeyang type, the selector 130 may select the reference table for the Taeyang type.

The reference table may be classified based on demographic information. The demographic information may include at least one of an age and a gender of a user.

The reference table may be formed based on an age. When a predetermined age is selected, reference tables for the Taeyang type, the Taeeum type, the Soyang type, and the Soeum type corresponding to the selected age may be provided. For example, ages may be classified into a category of ages 0 to 20, a category of ages 21 to 40, a category of ages 41 to 60, and a category of ages over 60. When the user is at an age 25 and corresponds to the Taeeum type, the selector 130 may select the category of ages 21 to 40, and then select a reference table for the Taeeum type from the category.

The reference table may be formed based on a gender. When a predetermined gender is selected, reference tables for the Taeyang type, the Taeeum type, the Soyang type, and the Soeum type corresponding to the selected gender may be provided.

The reference table may be formed based on an age and a gender. For example, ages may be classified into a category of ages 0 to 20, a category of ages 21 to 40, a category of ages 41 to 60, and a category of ages over 60. Additionally, each of the categories may be classified based on a gender. When the user is a male at an age 28 and corresponds to the Soyang type, the selector 130 may select a category of a male from the category of ages 21 to 40 and then, select a reference table for the Soyang type from the category.

The determiner 140 may determine a first constitutional disease pattern corresponding to the first deficiency and excess index and the first cold and heat condition index of the user among a plurality of constitutional disease patterns included in the first reference table. The first reference table may be a reference table corresponding to a first constitution among the reference tables and the first constitution may be a constitution of the user obtained through a classification based on the Sasang constitution.

The first reference table may correspond to the user demographic information. The user demographic information may include at least one of an age and a gender of the user.

In an example embodiment, the determiner 140 may determine a constitutional disease pattern having a minimum geometric distance from a first point corresponding to the first deficiency and excess index and the first cold and heat condition index among the plurality of constitutional disease patterns included in the first reference table, as the first constitutional disease pattern.

In another example embodiment, the determiner 140 may provide at least one second constitutional disease pattern having a greater geometric distance when compared to the first constitutional disease pattern as well as the first constitutional disease pattern having the minimum geometric distance from the first point. For example, the determiner 140 may provide a disease pattern B as well as a disease pattern A.

In still another example embodiment, the determiner 140 may provide the at least one second constitutional disease pattern by assigning a weight thereto. The weight may be inversely proportional to the geometric distance. For example, a point corresponding to the first deficiency and excess index and the first cold and heat condition index of the user in the first reference table may be referred to as the first point. When a distance between the disease pattern A and the first point is “3” and a distance between the disease pattern B and the first point is “7”, the determiner 140 may provide information indicating a 70% probability of the disease pattern A and a 30% probability of the disease pattern B.

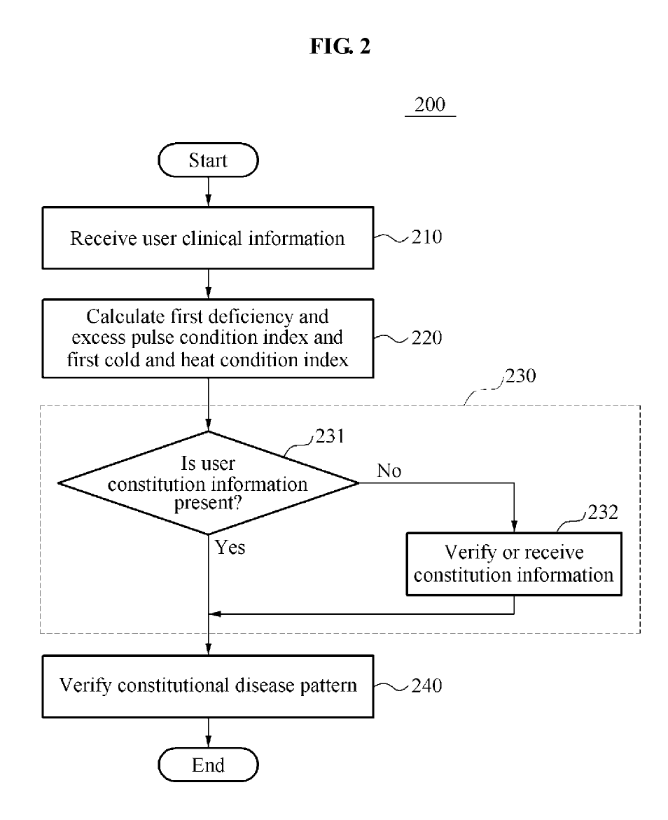

FIG. 2 is a flowchart illustrating a constitutional disease pattern determination method according to an example embodiment.

In operation 210, the constitutional disease pattern determination apparatus 100 receives user clinical information. The user clinical information may include first data indicating responses of the user to each of a plurality of first medical examination items for calculating a first deficiency and excess index. Also, the user clinical information may include second data indicating responses of the user to each of a plurality of second medical examination items for calculating a first cold and heat condition index.

The first medical examination items may include at least one of an appetite state, a digestion state, cold sweats, bowel movement regularity, a number of nocturias, a sleep quality, dry mouth, information associated with a pulse diagnosis, an asthenia examined from an abdominal diagnosis, and a subumbilical asthenia.

The second medical examination items may include at least one of an amount of sweat, a number of bowel movements, a feces shape, a number of urinations, a typical body temperature, a degree of cold sensitivity, a degree of heat sensitivity, an amount of drinking water, a drinking water temperature, a degree of thirst, information on a pulse diagnosis, and information on a tongue coating.

In operation 220, the first deficiency and excess index and the first cold and heat condition index are calculated based on the user clinical information.

The calculator 110 may calculate the first deficiency and excess index based on the first data classified into a deficiency condition, an intermediate condition, and a excess condition.

When a number of deficiency conditions is greater than a number of excess conditions, the calculator 110 may calculate a deficiency condition score as a ratio of a difference between the number of deficiency conditions and the number of excess conditions to a number of first medical examination items.

When the number of excess conditions is greater than the number of deficiency conditions, the calculator 110 may calculate a excess condition score as a ratio of a difference between the number of excess conditions and the number of deficiency conditions to the number of first medical examination items.

When the number of deficiency conditions is equal to the number of excess conditions, the calculator 110 may calculate the deficiency condition score and the excess condition score as an intermediate condition score.

The calculator 110 may calculate the first cold and heat condition index based on the second data classified into a cold condition, an intermediate condition, and a heat condition.

When a number of cold conditions is greater than a number of heat conditions, the calculator 110 may calculate a cold condition score as a ratio of a difference between the number of cold conditions and the number of heat conditions to a number of the second medical examination items.

When the number of heat conditions is greater than the number of cold conditions, the calculator 110 may calculate a heat condition score as a ratio of a difference between the number of heat conditions and the number of cold conditions to the number of second medical examination items.

When the number of cold conditions is equal to the number of heat conditions, the calculator 110 may calculate the cold condition score and the heat condition score as an intermediate condition score.

In operation 230, user constitution information is acquired.

In operation 231, whether the user constitution information is present in the constitutional disease pattern apparatus 100 is determined. When the user constitution information is present, the constitutional disease pattern of the user may be determined based on the first deficiency and excess index and the first cold and heat condition index calculated in operation 220.

When the user constitutional information is absent, in operation 232, the constitutional disease pattern determination apparatus 100 verifies or receives the user constitutional information.

In operation 240, the constitutional disease pattern of the user is verified. The constitutional disease pattern may be verified based on the first reference table corresponding to the first constitution indicating the constitution of the user among the reference tables classified based on the Sasang constitution. The first constitutional disease pattern corresponding to the first deficiency and excess index and the first cold and heat condition index of the user may be determined from among the plurality of constitutional disease patterns included in the first reference table.

In an example embodiment, when the first constitution is classified as a Taeeum type based on the Sasang constitution, the plurality of constitutional disease patterns included in the first reference table may include at least one of an Supraspinal Exterior pattern, Esophagus Cold pattern, a Dryness Heat pattern, Dryness Heat pattern with Yin Blood Consumption symptoms, and an Exterior Interior combined pattern.

In another example embodiment, when the first constitution is classified as a Soeum type based on the Sasang constitution, the plurality of constitutional disease patterns included in the first reference table may include at least one of a depression-manic, a Yang collapse, a greater Yin pattern, a lesser Yin pattern, and an Exterior Interior combined pattern.

In still another example embodiment, when the first constitution is classified as a Soyang type based on the Sasang constitution, the plurality of constitutional disease patterns included in the first reference table may include at least one of a Lesser Yang Wind Damage pattern, a Yin Depletion pattern, a Chest Heat congested pattern, a Yin Deficit Diurnal Heat pattern, and an Exterior Interior combined pattern.

In yet another example embodiment, when the first constitution is classified as a Taeyang type based on the Sasang constitution, the plurality of constitutional disease patterns included in the first reference table may include at least one of a Interior origin Small Intestine pattern accompanying Regurgitation, Interior origin Small Intestine pattern, and an Exterior Interior combined pattern.

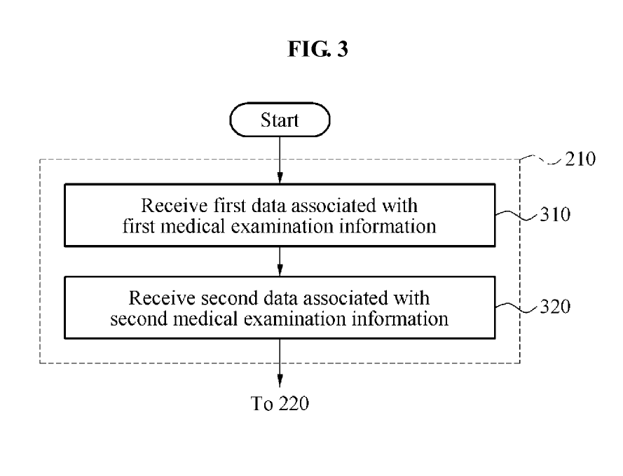

FIG. 3 is a diagram illustrating an operation of receiving clinical information of FIG. 2.

The operation of receiving clinical information includes operation 310 of receiving first data associated with first medical examination information and operation 320 of receiving second data associated with second medical examination information.

The clinical information may include the first data indicating responses of a user to each of a plurality of first medical examination items for calculating a first deficiency and excess index.

The first medical examination information may include at least one of an appetite state, a digestion state, cold sweats, bowel movement regularity, a number of nocturias, a sleep quality, a dry mouth, information on a pulse diagnosis, an asthenia examined from an abdominal diagnosis, and a subumbilical asthenia.

The information on the pulse diagnosis may include information on pulse conditions of “deficiency”, “excess”, “long”, “short”, “surging”, and “thready”.

The clinical information may include the second data indicating responses of the user to each of a plurality of second medical examination items for calculating the first cold and heat condition index.

The second medical examination information may include one of an amount of sweat, a number of bowel movements, a feces shape, a number of urinations, a typical body temperature, a degree of cold sensitivity, a degree of heat sensitivity, an amount of drinking water, a drinking water temperature, a degree of thirst, information on a pulse diagnosis, and information on a tongue coating.

The information on the pulse diagnosis may include information on pulse conditions of “floating”, “sunken”, “slow”, and “rapid”.

The information on the tongue coating may include information on tongue conditions of “yellow”, “white”, and “crimson”.

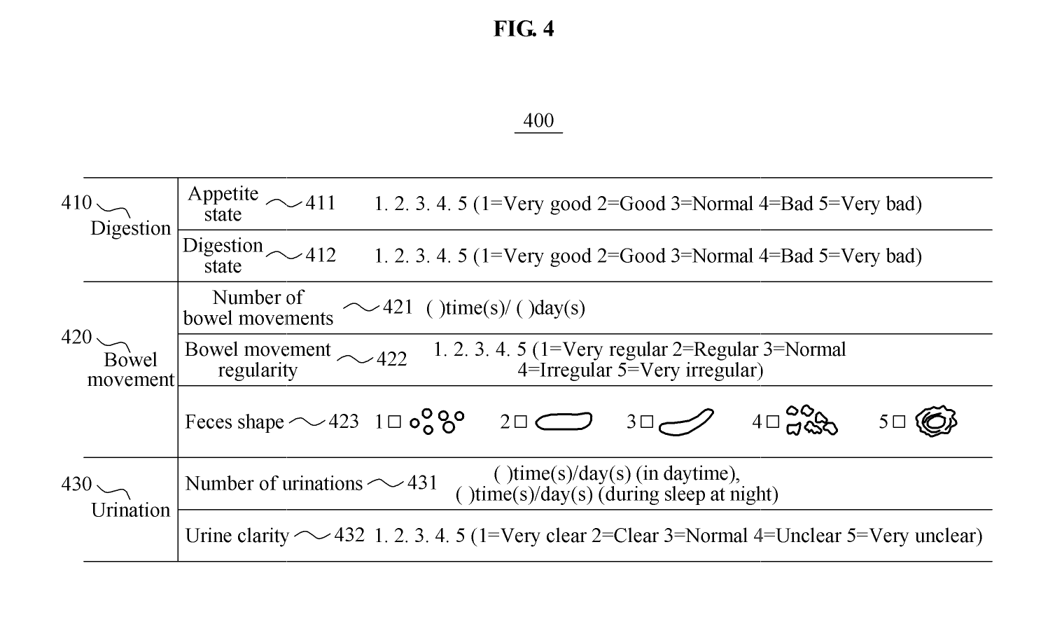

FIG. 4 is a diagram illustrating an example of medical examination information according to an example embodiment.

The medical examination information may include a first medical examination item for calculating a first deficiency and excess index. Also, the medical examination information may include a second medical examination item for calculating a first cold and heat condition index.

Referring to FIG. 4, a table 400 represents the medical examination information including an item 410, for example, digestion, an item 420, for example, a bowel movement, and an item 430, for example, urination.

A response to the medical examination information may be provided using a numerical value and a term determined based on a state of a user in advance.

Medical examination information on the digestion may include a medical examination item for calculating a deficiency and excess index.

The predetermined numerical value may be expressed by integers 1 through 5. For example, a response 411 may be an appetite state in the item 410 indicating the medical examination information on the digestion. The appetite state may be expressed using one of a “very good” scoring one point, a “good” scoring two points, a “normal” scoring three points, a “bad” scoring four points, and a “very bad” scoring five points.

In the medical examination information on the digestion, a response 412, for example, a digestion state may be expressed using numerical values similarly to the numerical values of the response 411.

The item 420 indicating medical examination information on the bowel movement may include medical examination information, for example, bowel movement regularity 422, for calculating the deficiency and excess index, and medical examination information, for example, a number of bowel movements 421 and a feces shape 423, for calculating a cold and heat condition index.

The number of bowel movements 421 may indicate the number of bowel movements in a day.

The bowel movement regularity 422 may be expressed using one of a “very regular” scoring a one point, a “regular” scoring two points, a “normal” scoring three points, an “irregular” scoring four points, and a “very irregular” scoring five points.

The feces shape 423 may be expressed by marking a corresponding shape among shapes shown in FIG. 4.

The item 430 indicating medical examination information on the urination may include information on a number of urinations 431 and urine clarity 432.

The number of urinations 431 may indicate the number of daytime urinations and the number of nighttime urinations.

The urine clarity 432 may be expressed using one of a “very clear” scoring one point, a “clear” scoring two points, a “normal” scoring three points, a “unclear” scoring four points, and a “very unclear” scoring five points.

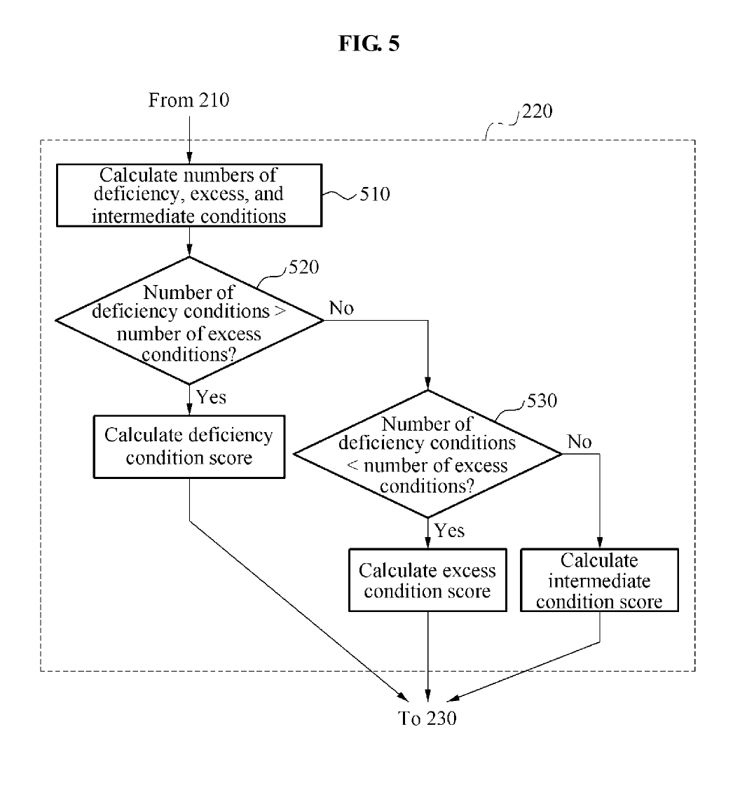

FIG. 5 is a diagram illustrating an operation of calculating a deficiency and excess index of FIG. 2.

In operation 510, the calculator 110 calculates a number of deficiency conditions, a number of excess conditions, and a number of intermediate conditions in clinical information on a user. The calculating may be performed based on first data classified into categories of a deficiency condition, an intermediate condition, and a excess condition. The calculating may be performed by counting a number of items selecting each of the categories of the deficiency condition, the intermediate condition, and the excess condition as responses.

In operation 520, whether the number of deficiency conditions is greater than the number of excess conditions is verified. When the number of deficiency conditions is greater than the number of excess conditions, the calculator 110 may calculate a deficiency condition score. The deficiency condition score may be expressed as a ratio of a difference between the number of deficiency conditions and the number of excess conditions to a number of first medical examination items. The first medical examination items may be medical examination items for calculating a deficiency and excess index. The deficiency condition score may be calculated using Equation 1.

$$\frac{( \begin{matrix}Number of deficiency conditions - \\ Number of excess conditions\end{matrix} )}{Number of first medical examination items} \times 100 \qquad [ Equation 1 ]$$

In operation 530, whether the number of excess conditions is greater than the number of deficiency conditions is verified. When the number of excess conditions is greater than the number of deficiency conditions, the calculator 110 may calculate a excess condition score. The excess condition score may be expressed as a ratio of a difference between the number of excess conditions and the number of deficiency conditions to the number of first medical examination items. The excess condition score may be calculated using Equation 2.

$$\frac{( \begin{matrix}Number of excess conditions - \\ Number of deficiency conditions\end{matrix} )}{Number of first medical examination items} \times 100 \qquad [ Equation 2 ]$$

When the number of excess conditions is not greater than the number of deficiency conditions, the number of excess conditions may be equal to the number of deficiency conditions. In this example, the calculator 110 may calculate the deficiency condition score and the excess condition score as an intermediate condition score. The intermediate condition score may be “0”. A value of “0” may indicate a center position between the deficiency condition and the excess condition in a reference table.

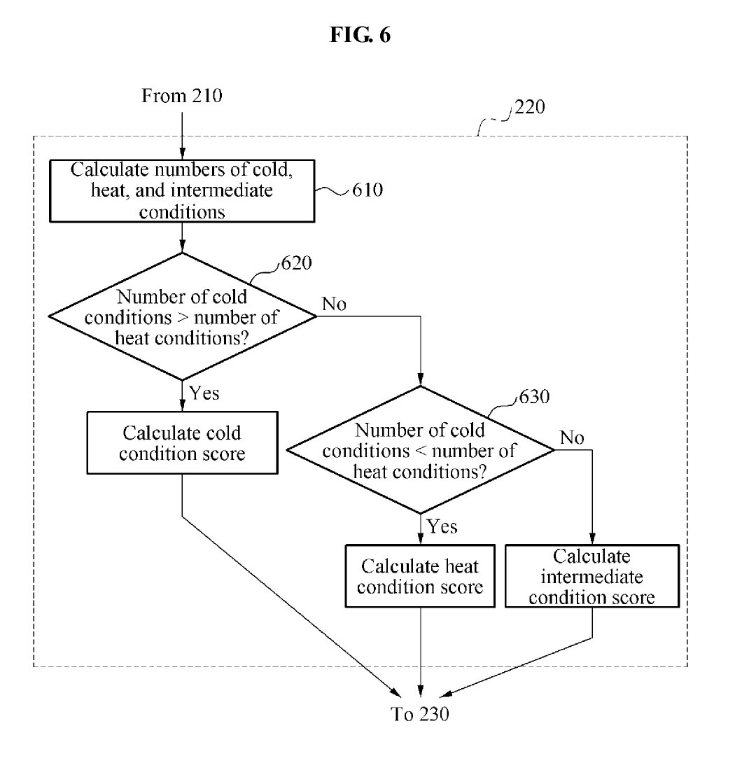

FIG. 6 is a diagram illustrating an operation of calculating a cold and heat condition index of FIG. 2.

In operation 610, the calculator 110 calculates a number of cold conditions, a number of intermediate conditions, and a number of heat conditions in clinical information on a user. The calculating may be performed based on second data classified into categories of a cold condition, an intermediate condition, and a heat condition. The calculating may be performed by counting a number of items selecting each of the categories of the cold condition, the intermediate condition, and the heat condition as responses.

In operation 620, whether the number of cold conditions is greater than the number of heat conditions is verified. When the number of cold conditions is greater than the number of heat conditions, the calculator 110 may calculate a cold condition score. The cold condition score may be expressed as a ratio of a difference between the number of cold conditions and the number of heat conditions to a number of second medical examination items. The second medical examination items may be medical examination items for calculating a cold and heat condition index. The cold condition score may be calculated using Equation 3.

$$\frac{( \begin{matrix}Number of cold conditions - \\ Number of heat conditions\end{matrix} )}{Number of second medical examination items} \times 100 \qquad [ Equation 3 ]$$

In operation 630, whether the number of heat conditions is greater than the number of cold conditions is verified. When the number of heat conditions is greater than the number of cold conditions, the calculator 110 may calculate a heat condition score. The heat condition score may be expressed as a ratio of a difference between the number of heat conditions and the number of cold conditions to the number of second medical examination items. The heat condition score may be calculated using Equation 4.

$$\frac{( \begin{matrix}Number of heat conditions - \\ Number of cold conditions\end{matrix} )}{Number of second medical examination items} \times 100 \qquad [ Equation 4 ]$$

When the number of heat conditions is not greater than the number of cold conditions, the number of heat conditions may be equal to the number of cold conditions. In this example, the calculator 110 may calculate the cold condition score and the heat condition score as an intermediate condition score. The intermediate condition score may be “0”. A value of “0” may indicate a center position between the cold condition and the heat condition in the reference table.

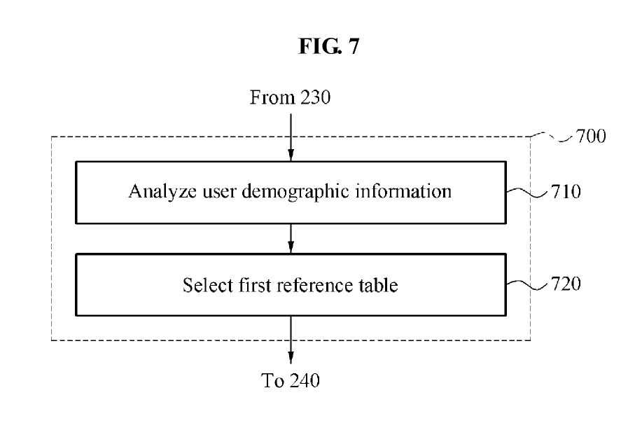

FIG. 7 is a diagram illustrating an operation of selecting a reference table corresponding to a user according to an example embodiment.

The constitutional disease pattern determination apparatus 100 may use user constitutional information to determine a constitutional disease pattern of the user. The constitutional disease pattern determination apparatus 100 may determine the constitutional disease pattern of the user based on a reference table corresponding to a constitution of the user.

When the user constitutional information is present in the constitutional disease pattern determination apparatus 100, the constitutional disease pattern determination apparatus 100 may determine the constitutional disease pattern of the user without determining the constitution of the user.

When the user constitutional information is absent in the constitutional disease pattern determination apparatus 100, the constitutional disease pattern determination apparatus 100 may determine the constitution of the user. Based on a result of the determining, the constitutional disease pattern determination apparatus 100 may determine the constitutional disease pattern of the user by mapping a deficiency and excess index and a cold and heat condition index of the user to a reference table.

Based on user demographic information, the reference table may be classified based on a Sasang constitution. The user demographic information may include at least one of an age and a gender of the user.

In operation 710, the selector 130 analyzes the user demographic information. In an example embodiment, the user demographic information may include at least one of an age and a gender of the user.

In operation 720, the selector 130 selects a first reference table in consideration of the constitution of the user. For example, the first reference table corresponding to the constitution of the user may be selected from among a plurality of reference tables corresponding to a plurality of constitutions classified based on the Sasang constitution.

In another example embodiment, the selector 130 may select the first reference table by combining the constitution of the user and the user demographic information. The constitutions may be classified into a Soeum type, a Soyang type, a Taeyang type, and a Taeeum type based on the Sasang constitution.

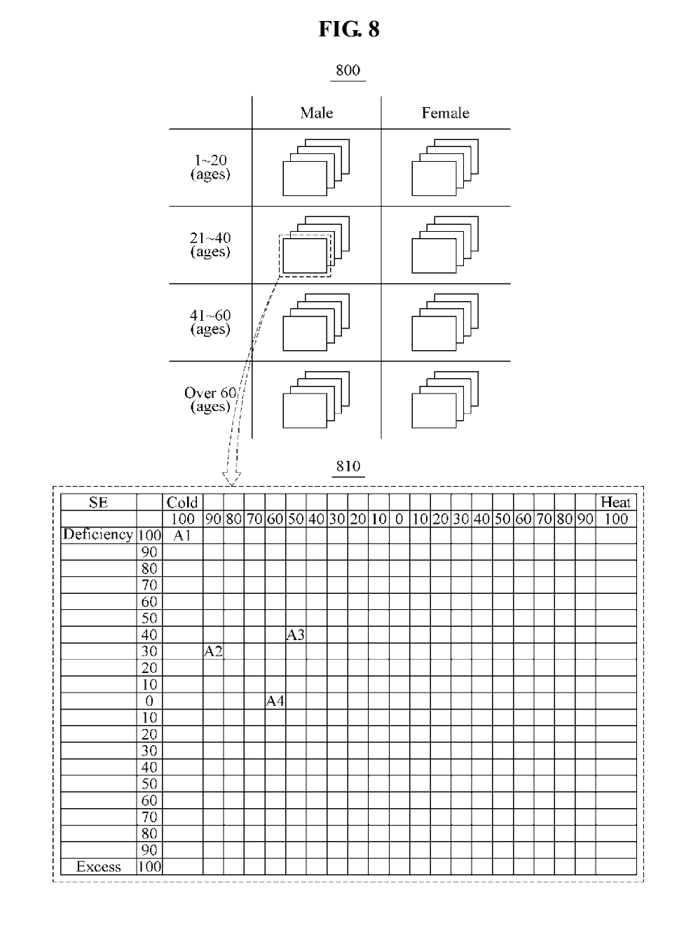

FIG. 8 is a diagram illustrating an example of selecting a reference table to correspond to user demographic information according to an example embodiment.

The first reference table may correspond to the user demographic information. The user demographic information may refer to information including at least one of an age and a gender of the user.

The user demographic information may be classified based on ages. The ages may be classified into four categories, for example, a category of ages 1 to 20, a category of ages 21 to 40, a category of ages 41 to 60, and a category of ages over 60.

The user demographic information may be classified based on genders. The genders may be classified into two categories, for example, a category of a male and a category of a female.

The user demographic information may be classified into eight categories based on an age and a gender of the user. A table 800 shows the eight categories with reference to FIG. 8.

For example, the user may be a male at an age 35 and correspond to a Taeyang type. In this example, the selector 130 may select the category of ages 21 to 40 and select a category of a male thereof. The selector 130 may select a reference table 810 of the Taeyang type from among reference tables of a Soeum type, a Soyang type, the Taeyang type, and a Taeeum type.

The reference table 810 may include constitutional disease patterns A1, A2, A3, and A4. The determiner 140 may compare a first deficiency and excess index and a first cold and heat condition index of the user to the reference table 810, and select a constitutional disease pattern of the user.

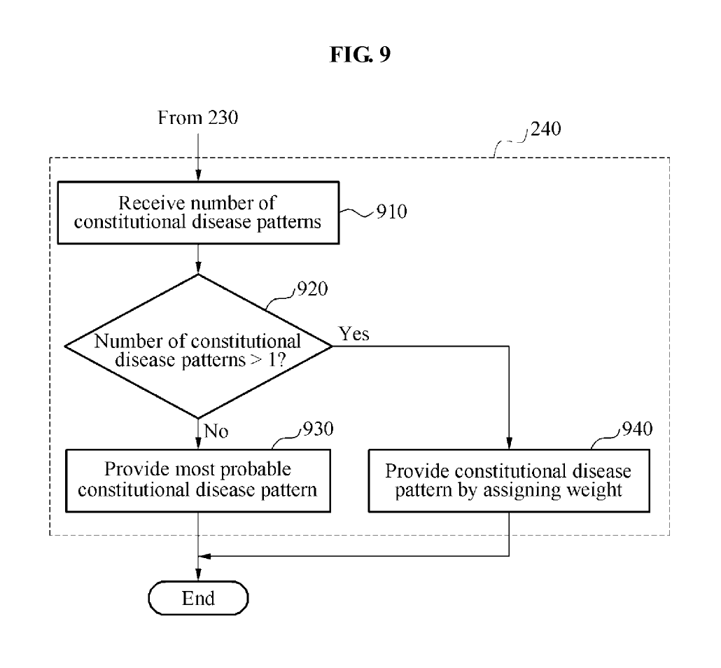

FIG. 9 is a diagram illustrating an operation of verifying a constitutional disease pattern of FIG. 2.

In an example embodiment, the determiner 140 may determine a first constitutional disease pattern based on a first reference table corresponding to a first constitution indicating a constitution of a user. The first constitutional disease pattern may be a constitutional disease pattern corresponding to a first deficiency and excess index and a first cold and heat condition index of the user among a plurality of constitutional disease patterns included in the first reference table.

In operation 910, the determiner 140 receives a number of constitutional disease patterns provided to the user.

In operation 920, whether the number of constitutional disease patterns is greater than “1” is verified.

In operation 930, the determiner 140 provides a constitutional disease pattern most probable to the user when the number of constitutional disease patterns is “1”.

In an example embodiment, the determiner 140 may determine the first constitutional disease pattern by selecting at least one of the plurality of constitutional disease patterns included in the first reference table. The first constitutional disease pattern may be a constitutional disease pattern having a minimum geometric distance from a first point corresponding to the first deficiency and excess index and the first cold and heat condition index of the user, among the plurality of constitutional disease patterns included in the first reference table.

In operation 940, the determiner 140 may provide the constitutional disease pattern to the user by assigning a weight when the number of constitutional disease patterns is greater than “1”.

In an example embodiment, the determiner 140 may determine at least one second constitutional disease pattern having a greater geometric distance when compared to the first constitutional disease pattern, among the plurality of constitutional disease patterns included in the first reference table.

In another example embodiment, when at least two constitutional disease patterns are provided, the determiner 140 may provide the constitutional disease pattern through an assignment of a weight. The weight may be inversely proportional to the geometric distance.

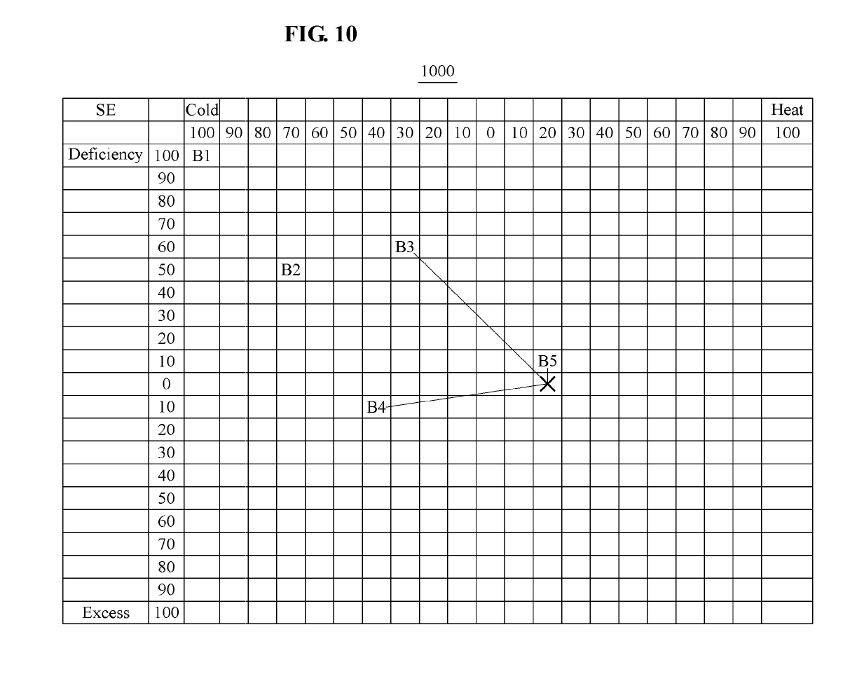

FIG. 10 is a diagram illustrating an example of determining a constitutional disease pattern according to an example embodiment.

Referring to FIG. 10, a deficiency and excess index may be zero points of an intermediate condition score, and a cold and heat condition index may be 20 points of a heat condition score. A point satisfying zero points of the intermediate condition score and 20 points of the heat condition score may be indicated as a first point.

In an example embodiment, a first reference table may be indicated by a table 1000. The first reference table may include constitutional disease patterns B1 through B5.

In the table 1000, the constitutional disease pattern B5 may be a constitutional disease pattern having a minimum geometric distance from the first point. The determiner 140 may provide information indicating a user corresponds to the constitutional disease pattern B5.

In another example embodiment, a constitutional disease pattern subsequently closest to the first point may be the constitutional disease pattern B4. Also, a constitutional disease pattern even subsequently closer to the first point may be the constitutional disease pattern B3. The determiner 140 may provide the constitutional disease patterns by assigning a weight. The weight may be inversely proportional to the geometric distance. The determiner 140 may provide information indicating the user has a 77% probability of the constitutional disease pattern B5, a 13% probability of the constitutional disease pattern B4, and a 10% probability of the constitutional disease pattern B3.

The units described herein may be implemented using hardware components and software components. For example, the hardware components may include microphones, amplifiers, band-pass filters, audio to digital converters, and processing devices. A processing device may be implemented using one or more general-purpose or special purpose computers, such as, for example, a processor, a controller and an arithmetic logic unit, a digital signal processor, a microcomputer, a field programmable array, a programmable logic unit, a microprocessor or any other device capable of responding to and executing instructions in a defined manner. The processing device may run an operating system (OS) and one or more software applications that run on the OS. The processing device also may access, store, manipulate, process, and create data in response to execution of the software. For purpose of simplicity, the description of a processing device is used as singular; however, one skilled in the art will appreciated that a processing device may include multiple processing elements and multiple types of processing elements. For example, a processing device may include multiple processors or a processor and a controller. In addition, different processing configurations are possible, such a parallel processors.

The software may include a computer program, a piece of code, an instruction, or some combination thereof, for independently or collectively instructing or configuring the processing device to operate as desired. Software and data may be embodied permanently or temporarily in any type of machine, component, physical or virtual equipment, computer storage medium or device, or in a propagated signal wave capable of providing instructions or data to or being interpreted by the processing device. The software also may be distributed over network coupled computer systems so that the software is stored and executed in a distributed fashion. In particular, the software and data may be stored by one or more computer readable recording mediums.

The methods according to the above-described embodiments may be recorded, stored, or fixed in one or more non-transitory computer-readable media that includes program instructions to be implemented by a computer to cause a processor to execute or perform the program instructions. The media may also include, alone or in combination with the program instructions, data files, data structures, and the like. The program instructions recorded on the media may be those specially designed and constructed, or they may be of the kind well-known and available to those having skill in the computer software arts. Examples of non-transitory computer-readable media include magnetic media such as hard disks, floppy disks, and magnetic tape; optical media such as CD ROM discs and DVDs; magneto-optical media such as optical discs; and hardware devices that are specially configured to store and perform program instructions, such as read-only memory (ROM), random access memory (RAM), flash memory, and the like. Examples of program instructions include both machine code, such as produced by a compiler, and files containing higher level code that may be executed by the computer using an interpreter. The described hardware devices may be configured to act as one or more software modules in order to perform the operations and methods described above, or vice versa.

Although a few embodiments of the present invention have been shown and described, the present invention is not limited to the described embodiments. Instead, it would be appreciated by those skilled in the art that changes may be made to these embodiments without departing from the principles and spirit of the invention, the scope of which is defined by the claims and their equivalents.

What is claimed is:

1. A constitutional disease pattern determination apparatus comprising:
a calculator to calculate a first deficiency and excess index and a first cold and heat condition index of a user based on clinical information on the user; and
a determiner to determine, based on a first reference table corresponding to a first constitution, a first constitutional disease pattern corresponding to the first deficiency and excess index and the first cold and heat condition index among a plurality of constitutional disease patterns included in the first reference table,
wherein the first constitution is a constitution classified to correspond to the user based on a Sasang constitution.

2. The apparatus of claim 1, wherein the first reference table corresponds to user demographic information, and the user demographic information comprises at least one of a gender and an age of the user.

3. The apparatus of claim 1, further comprising:
a selector to select the first reference table corresponding to the first constitution from among a plurality of reference tables corresponding to a plurality of constitutions classified based on the Sasang constitution.

4. The apparatus of claim 1, wherein the determiner determines a constitutional disease pattern having a minimum geometric distance from a first point corresponding to the first deficiency and excess index and the first cold and heat condition index among the plurality of constitutional disease patterns included in the first reference table, as the first constitutional disease pattern.

5. The apparatus of claim 4, wherein the determiner determines at least one second constitutional disease pattern having a greater geometric distance when compared to the first constitutional disease pattern among the plurality of constitutional disease patterns included in the first reference table, and provides the at least one second constitutional disease pattern.

6. The apparatus of claim 5, wherein the determiner provides the first constitutional disease pattern and the at least one second constitutional disease pattern through an assignment of a weight, and the weight is inversely proportional to the geometric distance.

7. The apparatus of claim 1, further comprising:
a verifier to verify the first constitution based on at least one of the clinical information and the demographic information on the user when identification information on the first constitution is absent.

8. The apparatus of claim 1, wherein the clinical information comprises first data indicating a response of the user to each of a plurality of first medical examination items for calculating the first deficiency and excess index, and second data indicating a response of the user to each of a plurality of second medical examination items for calculating the first cold and heat condition index.

9. The apparatus of claim 8, wherein the first medical examination items comprise at least one of an appetite state, a digestion state, cold sweats, bowel movement regularity, a number of nocturias, a sleep quality, a dry mouth, information on a pulse diagnosis, an asthenia examined from an abdominal diagnosis, and a subumbilical asthenia.

10. The apparatus of claim 9, wherein, based on the first data classified into a deficiency condition, an intermediate condition, and a excess condition, the calculator calculates a deficiency condition score corresponding to a ratio of a difference between a number of excess conditions and a number of deficiency conditions, to a number of first medical examination items when the number of deficiency conditions is greater than the number of excess conditions,
calculates a excess condition score corresponding to a ratio of a difference between the number of deficiency conditions and the number of excess conditions, to the number of first medical examination items when the number of excess conditions is greater than the number of deficiency conditions, and
calculates an intermediate condition score when the number of deficiency conditions is equal to the number of excess conditions.

11. The apparatus of claim 8, wherein the second medical examination items comprise at least one of an amount of sweat, a number of bowel movements, a feces shape, a number of urinations, a typical body temperature, a degree of cold sensitivity, a degree of heat sensitivity, an amount of drinking water, a drinking water temperature, a degree of thirst, information on a pulse diagnosis, and information on a tongue coating.

12. The apparatus of claim 11, wherein, based on the second data classified into a cold condition, an intermediate condition, and a heat condition, the calculator calculates a cold condition score corresponding to a ratio of a difference between a number of heat conditions and a number of cold conditions, to a number of second medical examination items when the number of cold conditions is greater than the number of heat conditions,
calculates a heat condition score corresponding to a ratio of a difference between the number of cold conditions and the number of heat conditions, to the number of second medical examination items when the number of heat conditions is greater than the number of cold conditions, and
calculates an intermediate condition score when the number of cold conditions is equal to the number of heat conditions.

13. The apparatus of claim 1, wherein when the first constitution is classified as a Taeeum type based on the Sasang constitution, the plurality of constitutional disease patterns included in the first reference table comprises at least one of an Supraspinal Exterior pattern, Esophagus Cold pattern, a Dryness Heat pattern, Dryness Heat pattern with Yin Blood Consumption symptoms, and an Exterior Interior combined pattern.

14. The apparatus of claim 1, wherein when the first constitution is classified as a Soeum type based on the Sasang constitution, the plurality of constitutional disease patterns included in the first reference table comprises at least one of a depression-manic, a Yang collapse, a greater Yin pattern, a lesser Yin pattern, and an Exterior Interior combined pattern.

15. The apparatus of claim 1, wherein when the first constitution is classified as a Soyang type based on the Sasang constitution, the plurality of constitutional disease patterns included in the first reference table comprises at least one of a Lesser Yang Wind Damage pattern, a Yin Depletion pattern, a Chest Heat congested pattern, a Yin Deficit Diurnal Heat pattern, and an Exterior Interior combined pattern.

16. The apparatus of claim 1, wherein when the first constitution is classified as a Taeyang type based on the Sasang constitution, the plurality of constitutional disease patterns included in the first reference table comprises at least one of a Interior origin Small Intestine pattern accompanying Regurgitation, Interior origin Small Intestine pattern, and an Exterior Interior combined pattern.

17. A method of determining a constitutional disease pattern of a user in a constitutional disease pattern determination apparatus, the method comprising:
calculating, by a calculator of the constitutional disease pattern determination apparatus, a first deficiency and excess index and a first cold and heat condition index of a user based on clinical information on the user; and
determining, by a determiner of the constitutional disease pattern determination apparatus, a first constitutional disease pattern corresponding to the first deficiency and excess index and the first cold and heat condition index among a plurality of constitutional disease patterns included in a first reference table corresponding to a first constitution, based on the first reference table,
wherein the first constitution is a constitution classified to correspond to the user based on a Sasang constitution.

18. The method of claim 17, wherein the first reference table corresponds to user demographic information, and the user demographic information comprises at least one of a gender and an age of the user.

19. The method of claim 17, wherein the determining comprises determining a constitutional disease pattern having a minimum geometric distance from a first point corresponding to the first deficiency and excess index and the first cold and heat condition index among the plurality of constitutional disease patterns included in the first reference table, as the first constitutional disease pattern.

20. The method of claim 17, the clinical information comprises first data indicating a response of the user to each of a plurality of first medical examination items for calculating the first deficiency and excess index, and second data indicating a response of the user to each of a plurality of second medical examination items for calculating the first cold and heat condition index.
